(19) United States
(12) Patent Application Publication
(10) Pub. No.: US 2023/0064504 A1
(43) Pub. Date: Mar. 2, 2023

(54) SYSTEM FOR COMBINED INTRAOPERATIVE ABERROMETRY AND OPTICAL COHERENCE TOMOGRAPHY

(71) Applicant: University of Miami, Miami, FL (US)

(72) Inventors: Marco Ruggeri, Miami, FL (US); Fabrice Manns, Miami, FL (US); Heather Durkee, Miami, FL (US)

(21) Appl. No.: 17/796,543

(22) PCT Filed: Jan. 29, 2021
(86) PCT No.: PCT/US2021/015724

§ 371 Date: Jul. 29, 2022

Related U.S. Application Data
(60) Provisional application No. 62/968,783, filed on Jan. 31, 2020.

(51) Int. Cl. A61B 3/10; A61B 3/00

(52) U.S. Cl. CPC A61B 3/1015; A61B 3/0025

(57) ABSTRACT

System for combined intraoperative aberrometry and optical coherence tomography (OCT). In an embodiment, the system comprises an OCT system, an aberrometer, a beam delivery system, and a beam splitter. The beam delivery system is configured to output a beam towards a target, wherein the beam has an outward path to the target and a return path after being reflected by the target. The beam splitter is positioned in the return path of the beam and configured to split the return path into a first path to the OCT system and a second path to the aberrometer. Thus, the OCT system and aberrometer can share a single beam delivery system.

### CROSS-REFERENCE TO RELATED APPLICATIONS

This application claims priority to U.S. Provisional Patent App. No. 62/968,783, filed on Jan. 31, 2020, which is hereby incorporated herein by reference as if set forth in full.

### BACKGROUND

### Field of the Invention

The embodiments described herein are generally directed to optical surgery, and, more particularly, to a system that optimally combines an intraoperative aberrometer with Optical Coherence Tomography (OCT) for guidance during surgery (e.g., cataract surgery).

### Description of the Related Art

Cataract surgery is the most common surgical procedure in the United States, with more than three million cases per year. By 2050, the number of Americans with cataracts is expected to double, with a subsequent increase in the number of surgeries. During cataract surgery, a cloudy lens inside the eye of a patient is removed and replaced with a clear artificial lens to restore the patient's vision. The visual outcome of cataract surgery is sometimes unpredictable, particularly in subjects who had prior LASIK surgery.

Currently, surgeons rely on eye biometry, performed before surgery, to make their best guess as to the correct intraocular lens (IOL) power and how to position the IOL inside the eye. This approach does not deliver the visual outcomes that patients expect from modern implants and instrumentation for cataract surgery (e.g., premium IOLs and femtosecond lasers). In particular, while IOL implantation generally produces good refractive outcomes (e.g., with 75% of patients within ±0.5D of the target refraction), the refractive outcomes remain unpredictable in a significant number of patients (e.g., 5%, or 175,000 per year, outside±1D).

Modern IOLs, including toric, aspheric, and multifocal IOLs, require fine control of IOL power and position. IOL power calculation, using standard IOL formulae, is particularly challenging and unpredictable in eyes with prior corneal refractive surgery, as a result of their altered corneal shapes. These challenges have generated the need for technology to intraoperatively guide IOL placement and verify refractive outcome during surgery.

### SUMMARY

Accordingly, a system is disclosed that enables surgeons to perform ocular biometry (i.e., eye measurements, e.g., using OCT and aberrometry) during surgery (e.g., cataract surgery), for example, to intraoperatively guide IOL placement and verify refractive outcomes during the surgery.

In an embodiment, the system comprises: an optical coherence tomography (OCT) system; an aberrometer; a beam delivery system configured to output a beam towards a target, wherein the beam has an outward path to the target and a return path after being reflected by the target; and a beam splitter positioned in the return path of the beam and configured to split the return path into a first path to the OCT system and a second path to the aberrometer.

The system may further comprise a control system communicatively coupled to one or both of OCT system and the aberrometer, wherein the control system comprises at least one hardware processor. The control system may be configured to: receive data from the aberrometer; and generate one or more measurements of refractive aberrations based on the data. The one or more measurements may comprise one or more of a sphere value, a cylinder value, or an axis value. The control system may be configured to: receive data from the OCT system; and generate at least one image based on the data.

The control system may be configured to operate in both a Shack-Hartmann (S-H) mode when the aberrometer implements S-H aberrometry, and a Laser-ray tracing (LRT) mode when the aberrometer implements LRT aberrometry. The control may be is configured to, when operating in the S-H mode, control the beam delivery system to deliver a stationary beam to the target. The control system may be configured to, when operating in the LRT mode: receive one or more parameters comprising one or both of a scan pattern or a number of rays; and control the beam delivery system to deliver a beam to the target according to the one or more parameters. The control system may be configured to, when operating in the LRT mode: acquire, as the data, an image of a retinal spot for each ray that is delivered to the target until a full scan of the target is completed; calculate a Zernike wavefront from the acquired images; and calculate the one or more measurements of refractive aberrations based on the Zernike wavefront.

The system may further comprise a pupil camera configured to capture an en face image of an eye for each acquisition by the aberrometer of an image of a retinal spot. The system may further comprise a ring illuminator. The ring illuminator may be turned on during image acquisition by the OCT system and turned off during sensing by the aberrometer. The system may further comprise an autorefractor.

The beam delivery system may be comprised in the OCT system. The OCT system may be configured to image an anterior segment of an eye. The OCT system and the aberrometer may be synchronized to operate in an interlaced pattern, such that the OCT system acquires images while the aberrometer is inactive, and the aberrometer performs sensing while the OCT system is inactive.

The system may further comprise a pupil camera that is turned on while the OCT system acquires images and turned off while the aberrometer performs sensing. The system may further comprise a ring illuminator that is turned on while the OCT system acquires images and turned off while the aberrometer performs sensing. The beam and aberrometer may be configured to acquire an image of a retinal spot with an angular extent of one to two degrees. The beam delivery system may be configured to output the beam at a power that is below a maximum safe exposure limit.

### BRIEF DESCRIPTION OF THE DRAWINGS

The details of the present invention, both as to its structure and operation, may be gleaned in part by study of the accompanying drawings, in which like reference numerals refer to like parts, and in which:

FIGS. 1A-1C illustrate alternative beam paths for OCT and aberrometry;

FIG. 2 illustrates an OCT image of a human eye, according to an embodiment;

FIG. 3 illustrates a combined system of OCT and aberrometry, according to an embodiment;

FIG. 4 illustrates a combined system of OCT and aberrometry integrated into a surgical microscope, according to an embodiment;

FIG. 5 illustrates a schematic of a combined system of OCT and aberrometry, according to an embodiment;

FIG. 6 is an image of a prototype of a combined system of OCT and aberrometry, according to an embodiment;

FIG. 7 illustrates an example calibration of a combined system of OCT and aberrometry, according to an embodiment;

FIG. 8 illustrates an example timing diagram for interlaced operations of a combined system of OCT and aberrometry, according to an embodiment;

FIG. 9 illustrates an example clinical evaluation of a combined system of OCT and aberrometry, according to an embodiment;

FIG. 10 illustrates an example processing system, by which one or more of the processes described herein, may be executed, according to an embodiment; and

FIGS. 11A-11D illustrate a combined system of OCT and aberrometry for installation under a surgical microscope, according to an embodiment.

### DETAILED DESCRIPTION

After reading this description, it will become apparent to one skilled in the art how to implement the invention in various alternative embodiments and alternative applications. However, although various embodiments of the present invention will be described herein, it is understood that these embodiments are presented by way of example and illustration only, and not limitation. As such, this detailed description of various embodiments should not be construed to limit the scope or breadth of the present invention as set forth in the appended claims.

One of the priorities of the corneal disease program of the National Eye Institute (NEI) is to address cataract surgery outcomes for the aging population of post-refractive surgery patients. These patients' altered corneas challenge the assumptions underlying current diagnostic technology.

With more than 3.5 million cases per year, cataract surgery is the most common procedure performed by ophthalmic surgeons. Cataract surgery generally involves removal of the crystalline lens and replacement of the lens with an IOL implant. Conventionally, the dioptric power of the IOL is selected using standard formulae that rely on preoperative ocular biometry. See, e.g., Holladay, “Refractive power calculations for intraocular lenses in the phakic eye,” Am J Ophthalmol 116, 63-66 (1993); Hoffmann et al., “Intraocular lens calculation for aspheric intraocular lenses,” Journal of cataract and refractive surgery 39, 867-872, doi:10.1016/j.jcrs.2012.12.037 (2013); Haigis, “Matrix-optical representation of currently used intraocular lens power formulas,” Journal of Refractive Surgery 25, 229-234 (2009); Holladay et al., “A three-part system for refining intraocular lens power calculations,” Journal of cataract and refractive surgery 14, 17-24 (1988); Retzlaff et al., “Development of the SRK/T intraocular lens implant power calculation formula,” Journal of cataract and refractive surgery 16, 333-340 (1990); and Hoffer, “The Hoffer Q formula: A comparison of theoretic and regression formulas,” Journal of cataract and refractive surgery 19, 700-712 (1993); which are all hereby incorporated herein by reference as if set forth in full. However, the availability of new advanced IOLs, which correct astigmatisms, and the low predictability of conventional biometry in eyes with prior refractive surgery, have generated a critical need for technology to intraoperatively guide IOL placement and verify the refractive outcome.

In embodiments, a system is disclosed that enables intraoperative ocular biometry by both OCT and aberrometry. For example, embodiments may sense ocular wavefront aberrations using the probing beam of an optical coherence tomography (OCT) system, such as an anterior segment OCT (AS-OCT) system. The disclosed system has the potential to produce a paradigm shift in cataract surgery by enabling precise image-guided IOL placement, as well as intraoperative ocular biometry and refraction. The combined intraoperative imaging and biometry may significantly improve the predictability of IOL calculations, especially in patients with prior refractive surgery. Advantageously, embodiments of the disclosed system can improve the overall visual outcomes of ocular surgeries (e.g., cataract surgery).

The disclosed approach enables the integration of both OCT and wavefront aberrometry within a surgical microscope, clinical biometry device, and/or other system. In an embodiment, instead of combining two separate devices, the system uses a new approach to integrate the two functionalities into a single device that uses a single, shared light source, beam delivery system, and control unit, with two separate detection channels, to generate both OCT images and aberration measurements. In other words, embodiments may comprise a consolidated multifunctional device for ocular surgery planning in a modular compact design that manufacturers can adapt to existing systems for rapid introduction into operating rooms. For example, the device can be integrated with existing ocular biometry systems.

The combined system with both OCT and aberrometry is, among other uses, suitable for studies on human subjects. In an embodiment, the system may be based on an existing extended depth spectral domain OCT (SD-OCT) system for anterior segment imaging. The system may use separate, interchangeable wavefront sensor modules (e.g., a Laser Ray Tracing module, and a Shack-Hartmann wavefront sensing module) integrated into an OCT system. A software platform running, for example, on a separate or integrated processing system, may synchronize and control data acquisition and compute wavefront refraction. The system can be tested and calibrated using an eye model or with respect to a clinical system.

1. Intraoperative Wavefront Aberrometry

Wavefront aberrometry measures aberrations in an eye based on changes or distortions in the wavefront of a beam of light that passes through the pupil and is reflected by the retina of the eye. There are currently two microscope-mounted intraoperative wavefront aberrometers, approved by the Food and Drug Administration (FDA), that enable refraction measurements during cataract surgery (i.e., ORA System™ by Alcon Management S.A. of Geneva, Switzerland, and Holos™ by Clarity Medical Systems, Inc.). See, e.g., Epitropoulos, “Visual and Refractive Outcomes of a Toric Presbyopia-Correcting Intraocular Lens,” Journal of ophthalmology 2016, 74581010, doi:10.1155/2016/74581010 (2016); Packer, “Effect of intraoperative aberrometry on the rate of postoperative enhancement: retrospective study,” Journal of cataract and refractive surgery 36, 747-755, doi:10.1016/j.jcrs.2009.11.029 (2010); Ianchulev et al., “Intraoperative refractive biometry for predicting intraocular lens power calculation after prior myopic refractive surgery,” Ophthalmology 121, 56-60, doi:10.1016/j.ophtha.2013.08.041 (2014); Canto et al., “Comparison of IOL power calculation methods and intraoperative wavefront aberrometer in eyes after refractive surgery,” Journal of refractive surgery (Thorofare, N.J.: 1995) 29, 484-489, doi:10.3928/1081597x-20130617-07 (2013); and “Intraoperative Wavefront Aberrometry: Wave of the Future?,” EyeNet Magazine, American Academy of Opthalmology, 38-43 (2013); which are all hereby incorporated herein by reference as if set forth in full. These devices have been evaluated for calculation of IOL power from aphakic refraction, measured after phacoemulsification, confirmation of the refractive outcome after IOL implantation, and guidance of toric IOL placement and corneal incisions to correct astigmatisms. Studies show that these current systems have shortcomings that limit their usefulness and impact, including, without limitation: their bulkiness and location under the microscope interferes with the surgical space (e.g., reducing the surgical working distance by about 30%); the need to mount and unmount the device in shared operating rooms interferes with the surgical workflow; the reproducibility of intraoperative refraction is low, affecting the reliability of IOL power calculations; alterations of the corneal and anterior chamber geometry during surgery limit the measurement reliability; and the need for repeated measurements and difficulties with alignment significantly increase surgical time.

2. Intraoperative Optical Coherence Tomography

Optical coherence tomography (OCT) is an imaging technique that uses low-coherence light to capture two-dimensional and three-dimensional images of an eye with micrometer-level resolution. Intraoperative OCT (iOCT) is used to guide or assist retinal and corneal surgeries. See, e.g., Ehlers et al., “The Prospective Intraoperative and Perioperative Ophthalmic Imaging with Optical Coherence Tomography (PIONEER) Study: 2-year results,” Am J Ophthalmol 158, 999-1007, doi:10.1016/j.ajo.2014.07.034 (2014); Steven et al., “Optimising deep anterior lamellar keratoplasty (DALK) using intraoperative online optical coherence tomography (iOCT),” Br J Ophthalmol 98, 900-904, doi:10.1136/bjophthalmol-2013-304585 (2014); Scorcia et al., “Anterior segment optical coherence tomography-guided big-bubble technique,” Ophthalmology 120, 471-476, doi:10.1016/j.ophtha.2012.08.041 (2013); Geerling et al., “Intraoperative 2-dimensional optical coherence tomography as a new tool for anterior segment surgery,” Archives of Ophthalmology 123, 253-257, doi:10.1001/archopht.123.2.253 (2005); Ide et al., “Intraoperative use of three-dimensional spectral-domain optical coherence tomography,” Ophthalmic Surgery Lasers and Imaging 41, 1050-254, doi:10.3928/15428877-20100303-15 (2010); Ruggeri et al., “Evaluation of a Surgical Microscope Interfaced SD-OCT system for Anterior Segment Surgery,” Investigative ophthalmology & visual science 56, 4088-4088 (2015); M et al., “Advanced Optical Technologies,” Vol. 2 233 (2013); Tao et al., “Image-Guided Modified Deep Anterior Lamellar Keratoplasty (DALK) Corneal Transplant Using Intraoperative Optical Coherence Tomography,” Investigative ophthalmology & visual science 56, 1966-1966 (2015); Tao et al., “Microscope-integrated intraoperative OCT with electrically tunable focus and heads-up display for imaging of ophthalmic surgical maneuvers,” Biomed Opt Express 5, 1877-1885, doi:10.1364/boe.5.001877 (2014); and Knecht et al., “Use of intraoperative fourier-domain anterior segment optical coherence tomography during descemet stripping endothelial keratoplasty,” Am J Ophthalmol 150, 360-365.e362, doi:10.1016/j.ajo.2010.04.017 (2010); which are all hereby incorporated herein by reference as if set forth in full. There are currently two FDA-approved iOCT systems (i.e., EnFocus™ by Leica Microsystems Inc. of Buffalo Grove, Ill., and Rescan700™ by Carl Zeiss AG of Oberkochen, Germany). Intraoperative anterior segment (AS) OCT (AS-OCT) provides the ability to visualize the state of the anterior chamber of the eye, and detects alterations in the shape of the anterior segment or misalignment during cataract surgery, which may affect the reliability of intraoperative refraction. iOCT also enables visualization, measurement, and intraoperative adjustment of the position of the IOL and prediction of the postoperative IOL position. See, e.g., Lytvynchuk et al., “Evaluation of intraocular lens position during phacoemulsification using intraoperative spectral-domain optical coherence tomography,” Journal of cataract and refractive surgery 42, 694-702, doi:10.1016/j.jcrs.2016.01.044 (2016); Hirnschall et al., “Predicting the postoperative intraocular lens position using continuous intraoperative optical coherence tomography measurements,” Investigative ophthalmology & visual science 54, 5196-5203, doi:10.1167/iovs.13-11991 (2013); and Hirnschall et al., “Using continuous intraoperative optical coherence tomography measurements of the aphakic eye for intraocular lens power calculation,” Br J Ophthalmol 99, 7-10, doi:10.1136/bjophthalmol-2013-304731 (2015); which are all hereby incorporated herein by reference as if set forth in full.

3. Combination of OCT and Aberrometry

In an embodiment, aberrometry and OCT are combined in a single device to significantly enhance surgical guidance during ocular surgery, such as cataract surgery. The mechanical and optical designs of current separate intraoperative aberrometers and OCT systems prevent their combined use during cataract surgery. Embodiments address this technological barrier using a solution that can be implemented in intraoperative microscope-integrated devices, as well as other types of devices. For the first time, aberrometry (e.g., wavefront aberrometry) and OCT (e.g., AS-OCT) can be integrated into a surgical microscope or other device. In an embodiment, the system uses a single light source and beam delivery system for both aberrometry and OCT.

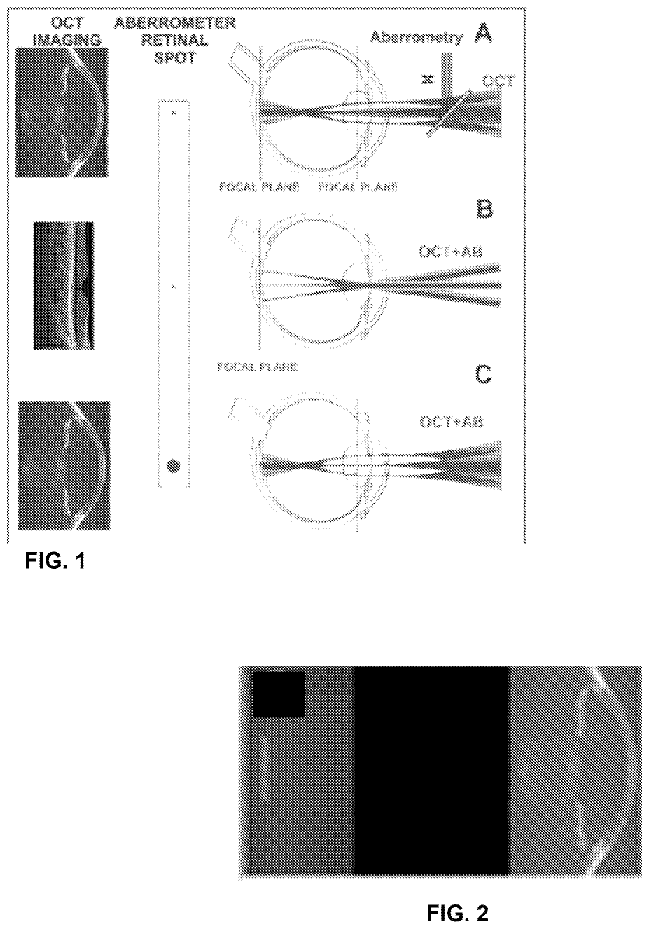

Systems with a slit-lamp-mounted Shack-Hartmann wavefront sensor in combination with AS-OCT have been developed for accommodation studies. These systems use two light sources with different wavelengths and two separate beam delivery systems. As illustrated in FIG. 1A, the OCT beam is focused in the anterior segment and scanned across the region of interest to acquire an image, whereas the aberrometer beam is focused on the retina and stationary. Integrating this standard dual-source approach, within the constrained environment of a surgical microscope, is challenging. It would result in a bulky attachment and would require the use of a large and specialized dichroic mirror to combine the two light sources.

Some Adaptive Optics OCT (AO-OCT) systems combine a retinal OCT system and wavefront sensor using a shared light source and beam delivery system. See, e.g., Hermann et al., “Adaptive-optics ultrahigh-resolution optical coherence tomography,” Optics letters 29, 2142-2144 (2004); Zhang et al., “Adaptive optics parallel spectral domain optical coherence tomography for imaging the living retina,” Opt Express 13, 4792-4811 (2005); and Zawadzki et al., “Adaptive-optics optical coherence tomography for high-resolution and high-speed 3D retinal in vivo imaging,” Opt Express 13, 8532-8546 (2005); which are all hereby incorporated herein by reference as if set forth in full. However, the AO-OCT in these systems is optimized to image a small region of the retina, as illustrated in FIG. 1B. They focus a narrow parallel beam on the retina. The beam geometry is optimized for both wavefront sensing and retinal imaging.

In contrast, in an embodiment of the disclosed system, the shared beam is focused in the anterior segment to optimize the OCT image quality, and scanned across the entire optical zone of the eye. The beam reaching or incident on the retina is defocused or divergent, as illustrated in FIG. 1C. The defocus is more pronounced in myopic eyes, which have a longer axial eye length. Notably, the size of the retinal spot produced by the disclosed system is larger than the size of the retinal spot produced by the systems illustrated in FIGS. 1A and 1B.

In an embodiment, the disclosed system, with combined OCT and aberrometry, produces one or both of:
(1) OCT images of the anterior segment or the whole eye, as illustrated in FIG. 2, with sufficient quality for clinical assessment and biometry. In an embodiment with a shared beam delivery system, the power returning from the eye may be split into a first channel or path to an OCT subsystem and a second channel or path to an aberrometer. The introduction of the aberrometry channel will produce a loss of power in the OCT channel, which in turn will reduce the signal-to-noise ratio of the OCT images. A trade-off exists between the minimum power required for reliable aberrometry, the minimum power required for OCT imaging, and the maximum power allowable to remain within safe exposure limits.
(2) A beam geometry that produces an acceptable trade-off between OCT image quality and wavefront aberrometry accuracy. The spot size in the anterior segment must be small to produce AS-OCT images with acceptable lateral resolution. At the same time, the retinal spot size must be less than the size of the isoplanatic patch to avoid wavefront measurement errors. See, e.g., Goncharov et al., “Reconstruction of the optical system of the human eye with reverse ray-tracing,” Opt Express 16, 1692-1703 (2008); and Nowakowski et al., “Investigation of the isoplanatic patch and wavefront aberration along the pupillary axis compared to the line of sight in the eye,” Biomed Opt Express 3, 1040-258, doi:10.1364/boe.3.0001040 (2012); which are both hereby incorporated herein by reference as if set forth in full. Thus, a trade-off exists between the small spot size required in the anterior segment for AS-OCT and the large-depth of focus required to maintain the retinal spot size within acceptable limits for wavefront aberrometry.

In an embodiment that uses the shared beam delivery configuration illustrated in FIG. 1C, a wavefront sensing (WFS) module for wavefront aberrometry is added to the existing OCT system with extended depth that is described in Ruggeri et al., “Imaging and full-length biometry of the eye during accommodation using spectral domain OCT with an optical switch,” Biomed. Opt. Express 3, 1506-1520, doi:10.1364/BOE.3.001506 (2012), which is hereby incorporated herein by reference as if set forth in full.

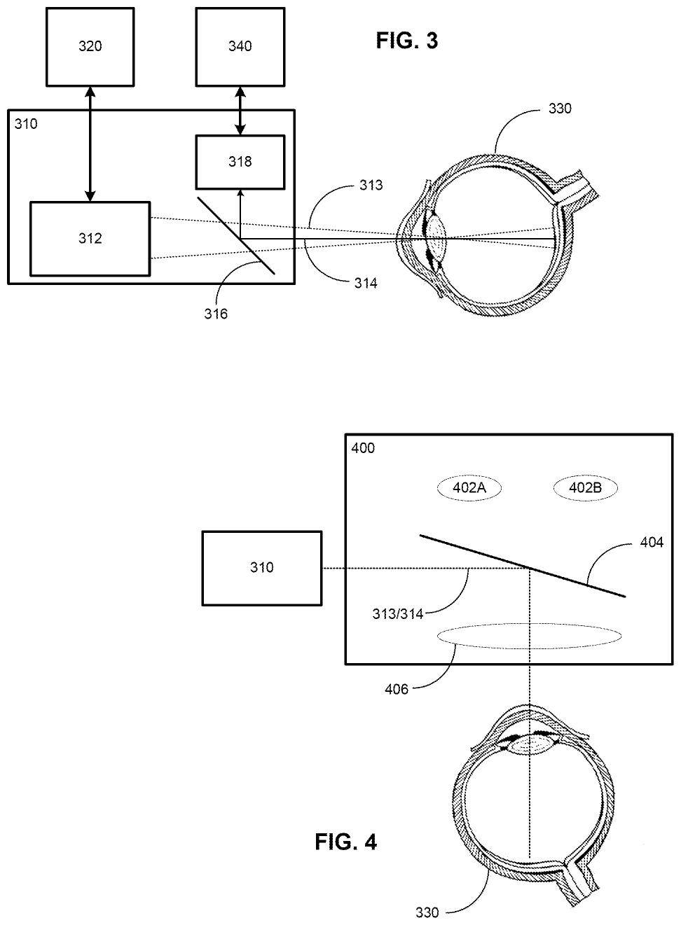

FIG. 3 illustrates the combined system of OCT and aberrometry, according to an embodiment. As illustrated, a combined delivery system 310 incorporates both a beam delivery system 312 and an aberrometer 318. Aberrometer 318 may comprise or consist of a WFS module. Combined delivery system 310 uses a single common light source 320, and aberrometer 318 communicates with a processing system 340. An existing OCT subsystem may comprise light source 320 and beam delivery system 312, which are shared with aberrometer 318. In other words, aberrometer 318 may be integrated with the OCT subsystem to utilize the OCT subsystem's existing light source 320 and beam delivery system 312. In this case, beam delivery system 312 produces an OCT beam 313 (e.g., AS-OCT beam) that passes into a patient's eye 330. OCT beam 313 is reflected off of the retina of eye 330, and this retinal reflection 314 is reflected off of beam splitter 316 to aberrometer 318. It should be understood that beam splitter 316 may split the reflected light into two paths, such that reflected light from the anterior segment of eye 330 is returned to the OCT subsystem along a first path (not shown), while retinal reflection 314 is passed to aberrometer 318 along a second path.

Different design approaches may be used for aberrometer 318, including, without limitation, Laser-ray tracing (LRT) and Shack-Hartmann (S-H). See, e.g., Molebny et al., “Retina ray-tracing technique for eye-refraction mapping,” 2971, 175-183 (1997); Navarro et al., “Aberrations and relative efficiency of light pencils in the living human eye,” Optometry and vision science: official publication of the American Academy of Optometry 74, 540-547 (1997); Smirnov et al., “Measurement of the wave aberration of the human eye,” Biofizika 6, 687-703 (1961); Moreno-Barriuso et al., “Ocular aberrations before and after myopic corneal refractive surgery: LASIK-induced changes measured with laser ray tracing,” Investigative ophthalmology & visual science 42, 1396-1403 (2001); Moreno-Barriuso et al., “Comparing laser ray tracing, the spatially resolved refractometer, and the Hartmann-Shack sensor to measure the ocular wave aberration,” Optometry and vision science: official publication of the American Academy of Optometry 78, 152-156 (2001); Liang et al., “Objective measurement of wave aberrations of the human eye with the use of a Hartmann-Shack wave-front sensor,” Journal of the Optical Society of America. A, Optics, image science, and vision 11, 1949-1957 (1994); and Diaz Santana Haro et al., “Single-pass measurements of the wave-front aberrations of the human eye by use of retinal lipofuscin autofluorescence,” Optics letters 24, 61-63 (1999); which are all hereby incorporated herein by reference as if set forth in full. The LRT approach is simpler in design. In this case, aberrometer 318 uses the galvanometer scanners of the OCT system for ray-tracing, and requires only a relay system and standard image sensor in a detection arm. The LRT approach may also provide a higher dynamic range than the S-H approach and requires less incident power. However, an advantage of the S-H approach is that it allows measurements at faster rates, since the beam does not need to be scanned across the measurement pupil.

Combined delivery system 310 may be mounted on a table-top support, such as a slit lamp mount, which can provide light source 320 and a connection to processing system 340. Alternatively, combined delivery system 310 may be integrated into a surgical microscope 400, as illustrated in FIG. 4, according to an embodiment. In this embodiment, OCT beam 313 and retinal reflection 314 may follow a path between combined delivery system 310 and eye 330 that is created by a dichroic beam splitter 404 between oculars 402 and objective lens 406. However, it should be understood that other arrangements are possible. Light source 320 may be provided by the light source of surgical microscope 400, which may utilize wide-field illumination.

The tables below summarize technical specifications of an embodiment of combined delivery system 310:

AS-OCT Subsystem

| Parameter | Specification | | Justification |
|---|---|---|---|
| Central | ~840 | nm | Standard OCT wavelength, and a |
| Wavelength | | | longer wavelength (e.g., 1300 nm) |
| | | | will result in higher attenuation, |
| | | | which is undesirable for aberrometry. |
| Axial | ~8 | μm (air) | Standard axial resolution is |
| Resolution | | | sufficient for biometry (super- |
| | | | luminescent diode (SLD) bandwidth |
| | | | with a full width at half maximum |
| | | | (FWHM) of ~50 nm). |
| Transverse | <100 | μm (air) | To detect anatomical changes with |
| Resolution | | | sufficient details and provide high |
| | | | image contrast. |
| Transverse | up to 16 mm | | For imaging the anterior segment |
| Scan Length | | | transversally from limbus to limbus. |
| Speed | 30,000 | Alines/s | Imaging speed of existing AS-OCT |
| | | | system is sufficient. |
| Axial Range | ~38.5 | mm (air) | To image the eye from cornea to |
| | | | retina for axial biometry. |

Wavefront Aberrometer

| Parameter | Specification | | Justification |
|---|---|---|---|
| Sphere Range | ±25 | D | To enable sphere measurements in |
| | | | the entire range of clinical interest. |
| Cylinder Range | ±8 | D | To enable astigmatism measurement |
| | | | in the entire range of clinical |
| | | | interest. |
| Precision | 0.125 | D | Standard precision of autorefractors. |
| Speed | >10 | Hz | To allow averaging of measurements |
| | | | in short time. |
| Angular Extent | 1-2 | degrees | To be within the isoplanatic patch |
| of Retina Spot | | | calculated at 840 nm. |
| Size | | | |

4. Shared Beam Delivery System

In an embodiment, beam delivery system 312 is designed and configured to be shared by AS-OCT and wavefront aberrometry, to create an OCT platform that enables the integration of one or more different modular aberrometers 318. An existing extended depth spectral domain AS-OCT system may be used as a starting point. Such a system produces images that range in depth from the cornea to the retina of an eye 330. A standard OCT beam delivery system may be used as beam delivery system 312, with a fiber collimator, dual-axis scanning mirrors, and an objective lens positioned at a focal distance from the scanning mirrors.

The numerical aperture (NA) of the OCT beam, produced by beam delivery system 312, is determined by the collimator and objective lens. Optical computer-assisted design (CAD) software, such as OpticStudio™ by Zemax, LLC of Kirkland, Wash., may be used to find the numerical aperture and lens combinations that provide the desired trade-off between AS-OCT lateral resolution, retinal spot size within the isoplanatic patch, and irradiance within the maximum permissible exposure (e.g., as established by International Organization for Standardization (ISO) 15004-2, American National Standards Institute Z136, etc.). For instance, a simulation using current AS-OCT beam geometry (e.g., collimator f=11 mm, objective f=100 mm) gives a focused spot diameter of 50 μm in the anterior segment and a retinal spot size of 300 μm for an eye length of 23 mm, corresponding to an angular extent of 1 degree, which is near the limit of the size of the isoplanatic patch.

Using dichroic mirrors and beam splitters, beam delivery system 312 may be combined with a pupil camera to facilitate centration. Beam delivery system 312 may also be combined with a fixation target. The combined system may be assembled with commercial optical cage mounts and mounted on a slit-lamp table. The mechanical setup may include a channel with a beam splitter 316 for attachment of aberrometer 318.

5. Aberrometers

In an embodiment, an aberrometer 318 is integrated within combined delivery system 310 to use the same beam delivery system 312 as the OCT subsystem. Aberrometer 318 may be modular and interchangeable, such that different aberrometers 318, which utilize different approaches (e.g., LRT or S-H), may be swapped in and out depending on preference or application. In other words, even after manufacture, combined delivery system 310 is not limited to a particular aberrometric approach, since a new approach can be implemented as a new modular aberrometer 318 and swapped in as needed or desired. To this end, combined delivery system 310 may comprise a socket into which aberrometer 318 can be connected and from which aberrometer 318 can be disconnected. Alternative connection designs may also be utilized. Processing system 340 may be configured to detect which aberrometer 318 has been connected to combined delivery system 310, and operate in a particular mode associated with the form of aberrometry (e.g., S-H or LRT) implemented by the detected aberrometer 318.

For both the S-H and LRT implementations of aberrometer 318, a standard 4f optical relay configuration may be used to image the pupil of eye 330. For the S-H implementation, the pupil may be imaged onto the lenslet array of a commercial off-the-shelf wavefront sensor (e.g., WFS150-5C by Thorlabs Inc. of Newark, N.J.). For the LRT implementation, the pupil may be imaged onto an objective lens that focuses retinal reflection 314 onto a commercial imaging sensor.

In both the S-H and LRT implementations, aberrometer 318 may be optically interfaced with beam delivery system 312 using beam splitter 316. Aberrometer 318 and beam splitter 316 may be assembled using commercial optical cage mounts. The mechanical assembly may be designed to enable easy connection of each interchangeable and modular aberrometer 318 to combined delivery system 310 and removal of each interchangeable and modular aberrometer 318 from combined delivery system 310.

The software for controlling combined delivery system 310 may provide a user-friendly interface. In an embodiment, the software was derived using LabVIEW™ by NI of Austin, Tex. In an embodiment, the software operates in different modes, depending on which aberrometer 318 is being used. For example, the software may operate in an S-H mode when aberrometer 318 implements S-H, and in an LRT mode when aberrometer implements LRT. In addition, the software may implement one or more, including potentially all, of the following operations:
(1) Scanning beam control. For an aberrometer 318 that implements S-H, the software controls beam delivery system 312 to deliver a stationary beam to eye 330. For an aberrometer 318 that implements LRT, the software receives user inputs, via the graphical user interface, specifying a scan pattern and/or number of rays to be delivered, and controls beam delivery system 312 to sequentially deliver each ray according to the specified scan pattern and/or number of rays.
(2) Acquisition. For an aberrometer 318 that uses S-H, the commercial wavefront sensors that come with LabVIEW™ drivers may be used to control the acquisition and record the spot patterns, wavefront data, and the refraction calculated from the wavefront data. For an aberrometer 318 that uses LRT, the imaging sensor acquires an image of the retinal spot for each ray that is delivered through the pupil until a full scan is completed, the images are transferred to processing system 340 for processing by the software which calculates the Zernike wavefront using standard modal decomposition algorithms, and the refraction is calculated from the Zernike wavefront using standard conversion formulae. In both cases, refraction may be calculated as sphere, cylinder, and axis. The sphere value indicates the lens power needed to see clearly, the cylinder value indicates the lens power needed to correct astigmatism, and the axis value indicates the angle between two meridians of an astigmatic eye. For both the S-H and LRT implementations of aberrometer 318, a pupil camera may capture and save an en face image of eye 330 at each acquisition.
(3) Display. The software displays the calculated refraction values (e.g., whether produced using S-H or LRT) and a real-time view from the pupil camera.

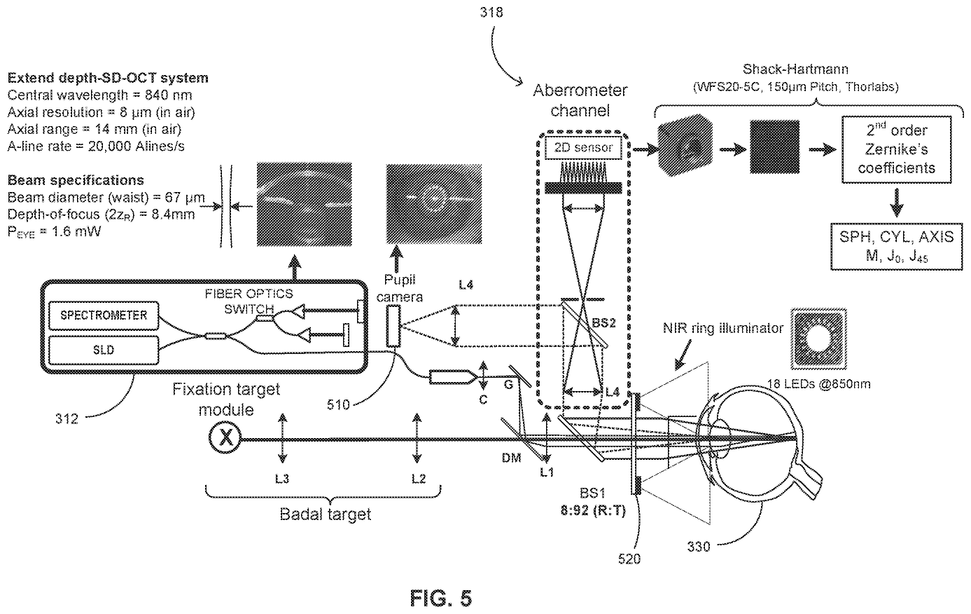

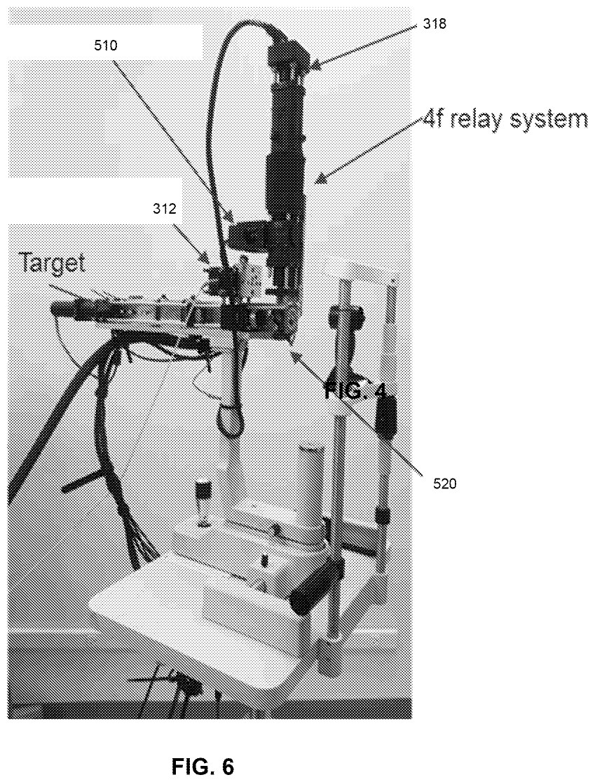

FIG. 5 illustrates a schematic of the disclosed system, according to an embodiment that is installed with an aberrometer 318 that implements S-H aberrometry. As illustrated, the system uses a single light source 320, as well as an integrated pupil camera 510 and a ring illuminator 520 for alignment of eye 330. FIG. 6 is an image of a prototype of the disclosed system mounted on a slit-lamp support, according to an embodiment.

6. Testing and Calibration

In an embodiment, the disclosed system is configured or calibrated ensure that the light beam, emitted by beam delivery system 312, is safe to the human eye. For example, the diameter of the OCT beam may be measured at different positions along the optical axis using a knife-edge technique to determine the beam geometry (e.g., waist diameter and divergence). This data may be used to calculate the exposure limit based on the ISO standard 15004-2. The power of light source 320 and/or beam delivery system 312 may then be adjusted to ensure that it remains below the exposure limit at all times while the system is active.

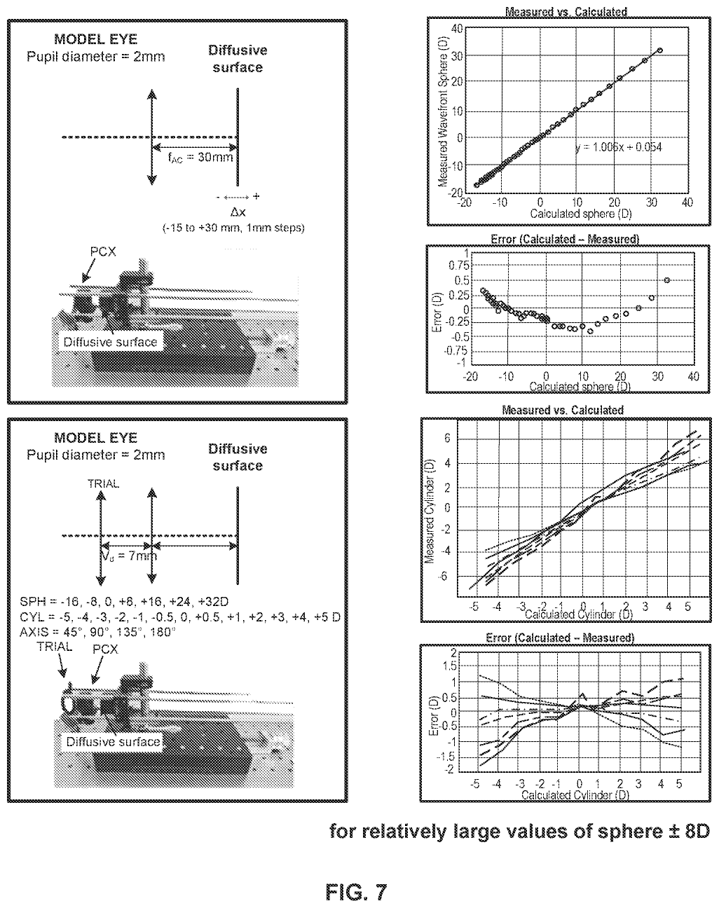

In an embodiment, each aberrometer 318 is calibrated using, for example, a custom-built eye model comprising a plano-convex lens (e.g., f=38 mm) and a plane Lambertian diffuse reflecting standard that models the retina of the human eye. The retina model may be mounted on a translation stage to allow the length of the eye model to be adjusted. The refractive error of the eye model can be adjusted from −20D to +30D by changing the position of the reflecting surface. For simulation of cylinder, cylindrical trial lenses may be mounted in front of the plano-convex lens. One or more of the following tests may be performed for each aberrometer 318:
(1) Calibration. The eye model may be mounted in front of the system, and calibration curves may be acquired. The calibration curves can be used to confirm the linearity of the measurement and quantify the measurement range. If needed, an aberrometer 318 may be fine-tuned by adjusting lens positions or using lenses with different focal lengths. FIG. 7 illustrates an example calibration, according to an embodiment.
(2) Repeatability. To determine the measurement precision of an aberrometer 318, repeated measurements of sphere, cylinder, and axis may be performed for multiple values of the refractive error of the eye model.
(3) Beam Optimization. The effect of the numerical aperture of the beam on aberrometer measurements and OCT image quality can be determined through experiments on the eye model and, for example, three or more human subjects. If the intensity in the S-H or LRT image sensor plane is insufficient to accurately calculate spot centroids, or if the OCT signal amplitude is insufficient to visualize the structures of interest, the numerical aperture of the OCT beam may be adjusted by selecting a different pair of collimator and objective lens (within relevant constraints) and/or splitting the ratio of beam splitter 316 used to separate the OCT and aberrometer beam paths until optimal performance is achieved.

7. Additional Features

In an embodiment, the focus of beam delivery system 312 is adjustable. This enables the beam geometry to be alternated when the system is switched from AS-OCT mode (FIG. 1B) to aberrometer mode (FIG. 1A). Tunable lenses and/or electromechanical systems for lens positioning can be used. See, e.g., Tao et al.; and Nankivil et al., “Handheld, rapidly switchable, anterior/posterior segment swept source optical coherence tomography probe,” Biomed Opt Express 6, 4516-4528, doi:10.1364/boe.6.004516 (2015), which is hereby incorporated herein by reference as if set forth in full.

In combined systems, it can be challenging to eliminate reflections from ocular surfaces or optical elements that interfere with the wavefront measurement. Thus, in an embodiment, polarization techniques, apertures, or slight decentration or tilt of the measurement beam can be used to eliminate reflections.

8. System Operations

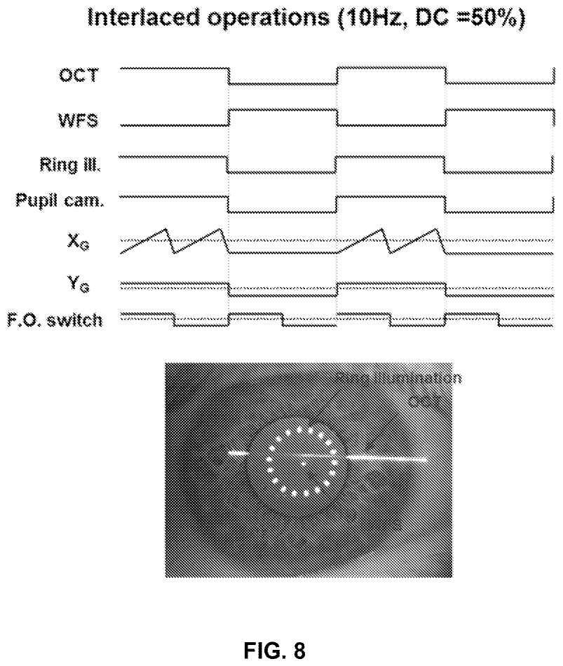

The operations of the disclosed system can be controlled so that the OCT system and aberrometer 318 operate one at a time. Alternatively, the OCT system and aberrometer 318 can be synchronized so that the wavefront measurements are acquired in an interlaced or alternating pattern with the OCT frame acquisitions. FIG. 8 illustrates an example timing diagram for such interlaced operations, according to an embodiment. The wavefront sensor measurements are acquired by aberrometer 318 (i.e., active) when the OCT system is not acquiring images (i.e., inactive), and images are acquired by the OCT system (i.e., active) while aberrometer 318 is not acquiring wavefront sensor measurements (i.e., inactive).

The timing diagram in FIG. 8 also illustrates the interlaced operations of pupil camera 510 and ring illuminator 520. For example, pupil camera 510 and ring illuminator 520 are both turned off during wavefront sensing by aberrometer 318, and both turned on during OCT acquisition. The OCT galvanometer signals, X_{G} and Y_{G}, are scanning during OCT acquisition and are steady and within the pupil during wavefront sensing. The bottom of FIG. 8, illustrates an example image of an eye, showing the different locations of the two-dimensional OCT scan and the wavefront sensing.

Despite the operating mode, the location of OCT imaging and wavefront sensing can be independently adjusted by setting different offset voltages on the signal driving the galvanometer mirrors. This capability enables, for example, the maximization of the OCT image quality and the accuracy of refraction measurements.

9. Clinical Evaluation

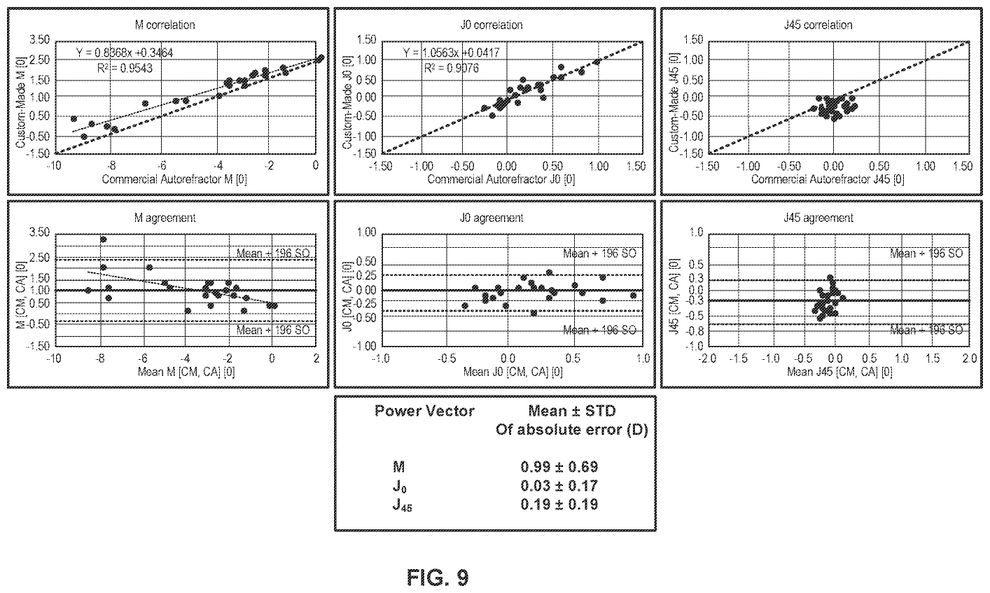

The ability of the aberrometer to produce refraction can be evaluated by comparing the measurements produced by the integrated aberrometer 318 with a clinical autorefractor (e.g., Topcon KR-800 by Lombart Instrument Co. of Norfolk, Va.). Measurements of human subjects can be acquired and compared with those obtained by the clinical autorefractor or aberrometer 318. FIG. 9 illustrates an example of a clinical evaluation, according to an embodiment. Power vectors acquired with the disclosed system were compared to those obtained with a clinical system. The correlation curves in the graphs can be used to calibrate the combined OCT-aberrometer with respect to a clinical system.

10. Example Processing System

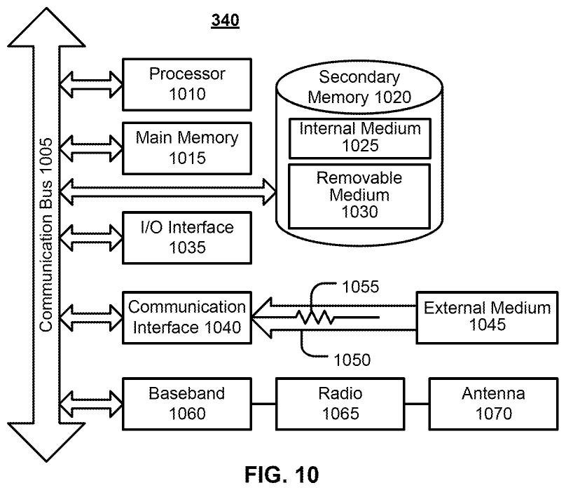

FIG. 10 is a block diagram illustrating an example processing system 340, according to an embodiment. System 340 may be used as or in conjunction with one or more of the functions, processes, or methods (e.g., to store and/or execute the software) described herein. System 340 can be a server or any conventional personal computer, or any other processor-enabled device that is capable of wired or wireless data communication. Other computer systems and/or architectures may be also used, as will be clear to those skilled in the art. It should be understood that, when system 340 is configured to control one or more aspects of combined delivery system 310 or other device, it may be referred to as a “control system.”

System 340 preferably includes one or more processors, such as processor 1010. Additional processors may be provided, such as an auxiliary processor to manage input/output, an auxiliary processor to perform floating-point mathematical operations, a special-purpose microprocessor having an architecture suitable for fast execution of signal-processing algorithms (e.g., digital-signal processor), a slave processor subordinate to the main processing system (e.g., back-end processor), an additional microprocessor or controller for dual or multiple processor systems, and/or a coprocessor. Such auxiliary processors may be discrete processors or may be integrated with processor 1010. Examples of processors which may be used with system 340 include, without limitation, the Pentium® processor, Core i7® processor, and Xeon® processor, all of which are available from Intel Corporation of Santa Clara, Calif.

Processor 1010 is preferably connected to a communication bus 1005. Communication bus 1005 may include a data channel for facilitating information transfer between storage and other peripheral components of system 340. Furthermore, communication bus 1005 may provide a set of signals used for communication with processor 1010, including a data bus, address bus, and/or control bus (not shown). Communication bus 1005 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

System 340 preferably includes a main memory 1015 and may also include a secondary memory 1020. Main memory 1015 provides storage of instructions and data for programs executing on processor 1010, such as one or more of the functions and/or modules discussed herein. It should be understood that programs stored in the memory and executed by processor 1010 may be written and/or compiled according to any suitable language, including without limitation C/C++, Java, JavaScript, Perl, Visual Basic, .NET, and the like. Main memory 1015 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

Secondary memory 1020 may optionally include an internal medium 1025 and/or a removable medium 1030. Removable medium 1030 is read from and/or written to in any well-known manner. Removable storage medium 1030 may be, for example, a magnetic tape drive, a compact disc (CD) drive, a digital versatile disc (DVD) drive, other optical drive, a flash memory drive, and/or the like.

Secondary memory 1020 is a non-transitory computer-readable medium having computer-executable code (e.g., disclosed software) and/or other data stored thereon. The computer software or data stored on secondary memory 1020 is read into main memory 1015 for execution by processor 1010.

In alternative embodiments, secondary memory 1020 may include other similar means for allowing computer programs or other data or instructions to be loaded into system 340. Such means may include, for example, a communication interface 1040, which allows software and data to be transferred from external storage medium 1045 to system 340. Examples of external storage medium 1045 may include an external hard disk drive, an external optical drive, an external magneto-optical drive, and/or the like. Other examples of secondary memory 1020 may include semiconductor-based memory, such as programmable read-only memory (PROM), erasable programmable read-only memory (EPROM), electrically erasable read-only memory (EEPROM), and flash memory (block-oriented memory similar to EEPROM).

As mentioned above, system 340 may include a communication interface 1040. Communication interface 1040 allows software and data to be transferred between system 340 and external devices (e.g. printers), networks, or other information sources. For example, computer software or executable code may be transferred to system 340 from a network server (e.g., platform 110) via communication interface 1040. Examples of communication interface 1040 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 340 with a network or another computing device. Communication interface 1040 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 1040 are generally in the form of electrical communication signals 1055. These signals 1055 may be provided to communication interface 1040 via a communication channel 1050. In an embodiment, communication channel 1050 may be a wired or wireless network, or any variety of other communication links. Communication channel 1050 carries signals 1055 and can be implemented using a variety of wired or wireless communication means including wire or cable, fiber optics, conventional phone line, cellular phone link, wireless data communication link, radio frequency (“RF”) link, or infrared link, just to name a few.

Computer-executable code (e.g., computer programs, such as the disclosed software) is stored in main memory 1015 and/or secondary memory 1020. Computer programs can also be received via communication interface 1040 and stored in main memory 1015 and/or secondary memory 1020. Such computer programs, when executed, enable system 340 to perform the various functions of the disclosed embodiments as described elsewhere herein.

In this description, the term “computer-readable medium” is used to refer to any non-transitory computer-readable storage media used to provide computer-executable code and/or other data to or within system 340. Examples of such media include main memory 1015, secondary memory 1020 (including internal memory 1025, removable medium 1030, and external storage medium 1045), and any peripheral device communicatively coupled with communication interface 1040 (including a network information server or other network device). These non-transitory computer-readable media are means for providing executable code, programming instructions, software, and/or other data to system 340.

In an embodiment that is implemented using software, the software may be stored on a computer-readable medium and loaded into system 340 by way of removable medium 1030, I/O interface 1035, or communication interface 1040. In such an embodiment, the software is loaded into system 340 in the form of electrical communication signals 1055. The software, when executed by processor 1010, preferably causes processor 1010 to perform one or more of the processes and functions described elsewhere herein.

In an embodiment, I/O interface 1035 provides an interface between one or more components of system 340 and one or more input and/or output devices. Example input devices include, without limitation, sensors, keyboards, touch screens or other touch-sensitive devices, biometric sensing devices, computer mice, trackballs, pen-based pointing devices, and/or the like. Examples of output devices include, without limitation, other processing devices, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and/or the like. In some cases, an input and output device may be combined, such as in the case of a touch panel display (e.g., in a smartphone, tablet, or other mobile device).

System 340 may also include optional wireless communication components that facilitate wireless communication over a voice network and/or a data network. The wireless communication components comprise an antenna system 1070, a radio system 1065, and a baseband system 1060. In system 340, radio frequency (RF) signals are transmitted and received over the air by antenna system 1070 under the management of radio system 1065.

In an embodiment, antenna system 1070 may comprise one or more antennae and one or more multiplexors (not shown) that perform a switching function to provide antenna system 1070 with transmit and receive signal paths. In the receive path, received RF signals can be coupled from a multiplexor to a low noise amplifier (not shown) that amplifies the received RF signal and sends the amplified signal to radio system 1065.

In an alternative embodiment, radio system 1065 may comprise one or more radios that are configured to communicate over various frequencies. In an embodiment, radio system 1065 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 1065 to baseband system 1060.

If the received signal contains audio information, then baseband system 1060 decodes the signal and converts it to an analog signal. Then the signal is amplified and sent to a speaker. Baseband system 1060 also receives analog audio signals from a microphone. These analog audio signals are converted to digital signals and encoded by baseband system 1060. Baseband system 1060 also encodes the digital signals for transmission and generates a baseband transmit audio signal that is routed to the modulator portion of radio system 1065. The modulator mixes the baseband transmit audio signal with an RF carrier signal, generating an RF transmit signal that is routed to antenna system 1070 and may pass through a power amplifier (not shown). The power amplifier amplifies the RF transmit signal and routes it to antenna system 1070, where the signal is switched to the antenna port for transmission.

Baseband system 1060 is also communicatively coupled with processor 1010, which may be a central processing unit (CPU). Processor 1010 has access to data storage areas 1015 and 1020. Processor 1010 is preferably configured to execute instructions (i.e., computer programs, such as the disclosed software) that can be stored in main memory 1015 or secondary memory 1020. Computer programs can also be received from baseband processor 1060 and stored in main memory 1010 or in secondary memory 1020, or executed upon receipt. Such computer programs, when executed, enable system 340 to perform the various functions of the disclosed embodiments.

11. Under-Microscope Embodiment

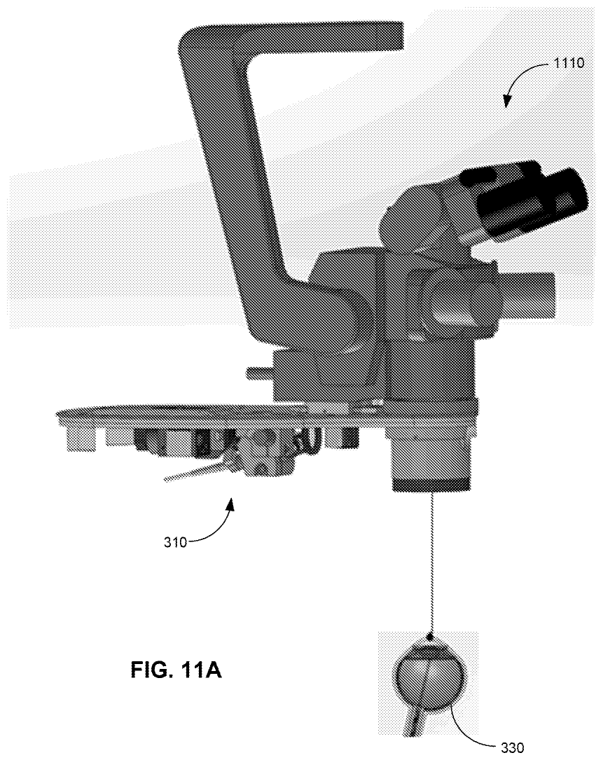

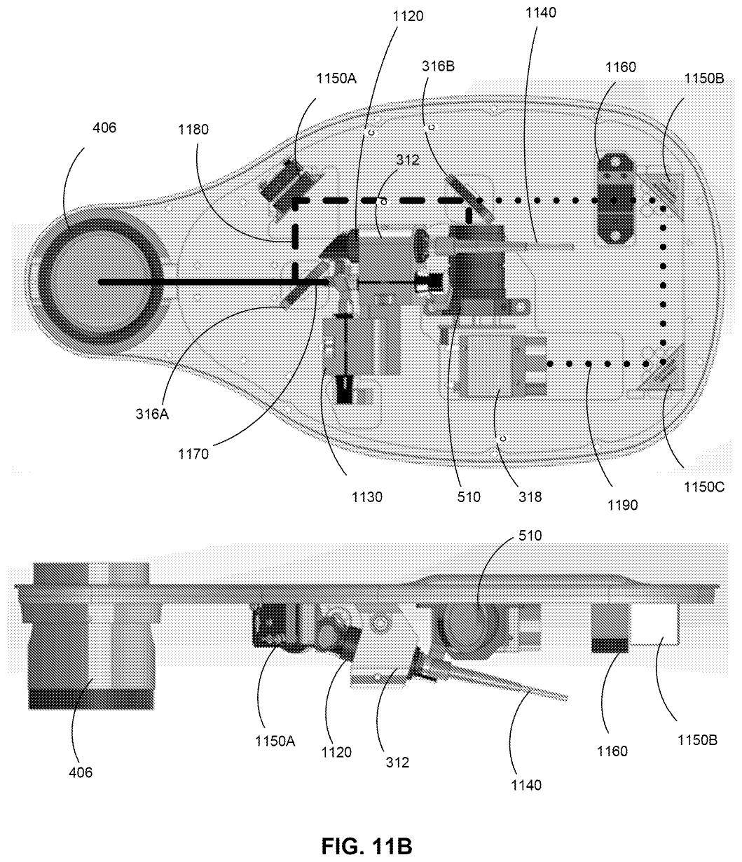

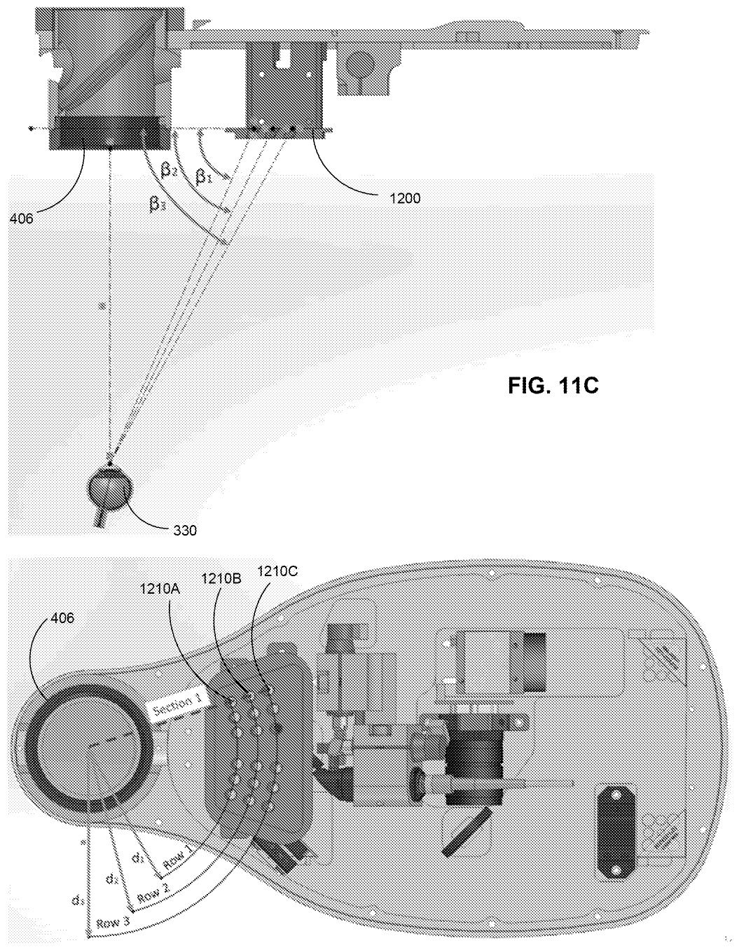

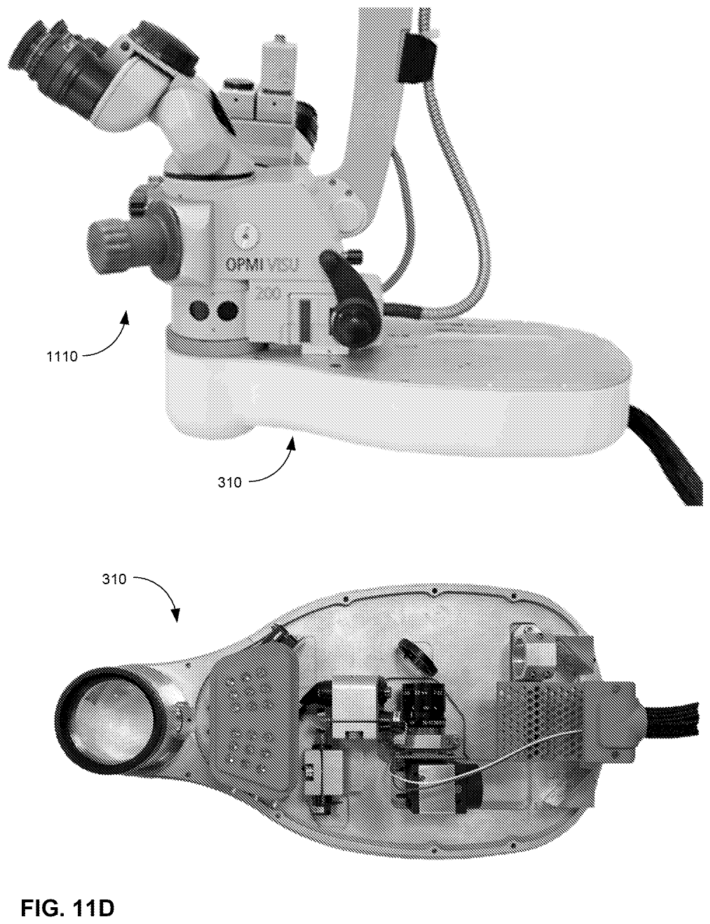

In a preferred embodiment, combined delivery system 310 is integrated into a surgical microscope. However, in an alternative embodiment, combined delivery system 310 may be a modular component that is attached underneath a surgical microscope. FIG. 11A illustrates a combined delivery system 310 attached underneath a surgical microscope 1100 (i.e., between surgical microscope 1100 and the surgical space), according to an embodiment. In the illustrated embodiment, the cover of combined delivery system 310 has been removed to illustrate the internal components.

FIG. 11B illustrates a bottom and side view of combined delivery system 310, according to an embodiment. As illustrated, combined delivery system 310 comprises an extension of objective lens 406 of surgical microscope 1100, beam delivery system 312 with collimator 1120, galvanometer mirrors 1130, and OCT light 1140 (e.g., coupled to light source 320 via optical fiber), beam splitters 316A and 316B, aberrometer 318, folding mirrors 1150, a lens 1160 of a 4f system, and a pupil camera 510. A beam is produced by beam delivery system 312 and passed through objective lens 406 to a target. The reflected beam from the target, as captured by the extension of objective lens 406, is split by beam splitter 316A into a first path 1170 to the OCT system and a second path 1180. The returned beam in second path 1180 is reflected off of folding mirror 1150A, and then split by beam splitter 316B into a third path to pupil camera 510 and a fourth path 1190. The returned beam in fourth path 1190 passes through lens 1160, and is reflected off of folding mirror 1150B and 1150C to aberrometer 318. In other words, the reflected beam from the target is split into various paths that provide a single reflected beam to each of the OCT system, pupil camera 510, and aberrometer 318.

FIG. 11C illustrates side and bottom views of an arc illuminator 1200, according to an embodiment. Arc illuminator 1200 may be used instead of ring illuminator 520 to illuminate eye 330 of a patient. Arc illuminator may comprise a plurality of rows 1210 of lights (e.g., LEDs) that are oriented towards a target of objective lens 406. Each LED may emit light with a wavelength of 850 nm. Each row 1210 of lights may be oriented at a different angle and radius with respect to objective lens 406 so that they are all focused on a single arc on the target (e.g., eye 330). For example, row 1210A may be oriented at an angle β₁ and radius d₁ with respect to objective lens 406, row 1210B may be oriented at an angle β₂ and radius d₂ with respect to objective lens 406, and row 1210C may be oriented at an angle β₃ and radius d₃ with respect to objective lens 406. While three rows 1210 are illustrated with six LEDs each (eighteen LEDs total), it should be understood that arc illuminator 1200 may comprise any number of rows 1210 and any number of LEDs or other lights per row 1210, including different numbers of lights per row 1210.

FIG. 11D illustrates a perspective view and bottom view of a prototype of combined delivery system 310 for installation under a surgical microscope 1110, according to an embodiment. In the perspective view, combined delivery system 310 is shown with its cover installed.

The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

What is claimed is:

1. A system comprising:
an optical coherence tomography (OCT) system;
an aberrometer;
a beam delivery system configured to output a beam towards a target, wherein the beam has an outward path to the target and a return path after being reflected by the target; and
a beam splitter positioned in the return path of the beam and configured to split the return path into a first path to the OCT system and a second path to the aberrometer.

2. The system of claim 1, further comprising a control system communicatively coupled to one or both of OCT system and the aberrometer, wherein the control system comprises at least one hardware processor.

3. The system of claim 2, wherein the control system is configured to:
receive data from the aberrometer; and
generate one or more measurements of refractive aberrations based on the data.

4. The system of claim 3, wherein the one or more measurements comprise one or more of a sphere value, a cylinder value, or an axis value.

5. The system of claim 2, wherein the control system is configured to:
receive data from the OCT system; and
generate at least one image based on the data.

6. The system of claim 2, wherein the control system is configured to operate in both a Shack-Hartmann (S-H) mode when the aberrometer implements S-H aberrometry, and a Laser-ray tracing (LRT) mode when the aberrometer implements LRT aberrometry.

7. The system of claim 6, wherein the control system is configured to, when operating in the S-H mode, control the beam delivery system to deliver a stationary beam to the target.

8. The system of claim 6, wherein the control system is configured to, when operating in the LRT mode:
receive one or more parameters comprising one or both of a scan pattern or a number of rays; and
control the beam delivery system to deliver a beam to the target according to the one or more parameters.

9. The system of claim 8, wherein the control system is configured to, when operating in the LRT mode:
acquire, as the data, an image of a retinal spot for each ray that is delivered to the target until a full scan of the target is completed;
calculate a Zernike wavefront from the acquired images; and
calculate the one or more measurements of refractive aberrations based on the Zernike wavefront.

10. The system of claim 1, further comprising a pupil camera configured to capture an en face image of an eye for each acquisition by the aberrometer of an image of a retinal spot.

11. The system of claim 1, further comprising a ring illuminator.

12. The system of claim 11, wherein the ring illuminator is turned on during image acquisition by the OCT system and turned off during sensing by the aberrometer.

13. The system of claim 1, further comprising an autorefractor.

14. The system of claim 1, wherein the beam delivery system is comprised in the OCT system.

15. The system of claim 1, wherein the OCT system is configured to image an anterior segment of an eye.

16. The system of claim 1, wherein the OCT system and the aberrometer are synchronized to operate in an interlaced pattern, such that the OCT system acquires images while the aberrometer is inactive, and the aberrometer performs sensing while the OCT system is inactive.

17. The system of claim 16, further comprising a pupil camera that is turned on while the OCT system acquires images and turned off while the aberrometer performs sensing.

18. The system of claim 16, further comprising a ring illuminator that is turned on while the OCT system acquires images and turned off while the aberrometer performs sensing.

19. The system of claim 1, wherein the beam and aberrometer are configured to acquire an image of a retinal spot with an angular extent of one to two degrees.

20. The system of claim 1, wherein the beam delivery system is configured to output the beam at a power that is below a maximum safe exposure limit.
